(19) United States
(12) Patent Application Publication
(10) Pub. No.: US 2021/0186996 A1
(43) Pub. Date: Jun. 24, 2021

(54) METHODS AND COMPOSITIONS FOR TREATING MULTIPLE SCLEROSIS

(71) Applicant: Elysium Health, Inc., New York, NY (US)

(72) Inventors: Eric Marcotulli, New York, NY (US); Dan Alminana, New York, NY (US); Mark Morris, New York, NY (US); Ryan Dellinger, Azusa, CA (US)

(21) Appl. No.: 16/768,299

(22) PCT Filed: Nov. 30, 2018
(86) PCT No.: PCT/US18/63202

§ 371 Date: May 29, 2020

Related U.S. Application Data
(60) Provisional application No. 62/593,601, filed on Dec. 1, 2017.

(51) Int. Cl. A61K 31/706; A61K 31/09; A61K 45/06; A61K 31/568; A61K 9/00; A61K 9/20; A61K 9/48

(52) U.S. Cl. CPC A61K 31/706; A61K 31/09; A61K 45/06; A61K 9/48; A61K 9/0053; A61K 9/20; A61K 31/568

(57) ABSTRACT

Provided herein are methods and compositions related to treating and/or preventing multiple sclerosis in a subject by administering to the subject (e.g., orally administering to the subject) a composition comprising nicotinamide riboside and/or pterostilbene.

### CROSS-REFERENCE TO RELATED APPLICATIONS

This application claims the benefit of U.S. Provisional Application No. 62/593,601, filed on Dec. 1, 2017; hereby incorporated by reference in its entirety.

### BACKGROUND

Multiple sclerosis (MS) is a chronic, often debilitating disease affecting the central nervous system (brain and spinal cord). MS affects more than 1 million people worldwide and is the most common neurological disease among young adults, particularly women. The exact cause of MS is still unknown. MS is an autoimmune disease in which myelin sheaths surrounding neuronal axons are destroyed. This condition can cause weakness, impaired vision, loss of balance, and poor muscle coordination.

MS takes several forms, with new symptoms either occurring in isolated attacks (relapsing forms) or building up over time (progressive forms). Between attacks, symptoms may disappear completely; however, permanent neurological problems often occur, especially as the disease advances.

MS takes several forms, with new symptoms either occurring in isolated attacks (relapsing forms) or building up over time (progressive forms). Between attacks, symptoms may disappear completely; however, permanent neurological problems often occur, especially as the disease advances.

There is no cure for multiple sclerosis. However, the development of new and novel treatments can help speed recovery from attacks, modify the course of the disease and manage symptoms.

### SUMMARY

In some aspects, the methods and compositions disclosed herein relate to treating autoimmune diseases, such as multiple sclerosis. Provided herein are methods and compositions related to treating multiple sclerosis and/or for treating and/or preventing a symptom or symptoms of multiple sclerosis, by administering to the subject (e.g., orally administering to the subject) a composition comprising a compound of Formula I or Formula II (e.g., nicotinamide riboside), and/or a compound of Formula III (e.g., pterostilbene).

The symptom may be fatigue, walking (gait) difficulties, numbness or tingling, spasticity, weakness, vision problems, dizziness or vertigo, bladder dysfunction, constipation, loss of control of bowels, chronic pain, depression, mood changes, dysarthria, dysphonia, dysphagia, seizures, tremor, pruritus, headache, hearing loss, and problems with respiration.

The multiple sclerosis may be relapsing-remitting multiple sclerosis, secondary-progressive multiple sclerosis, primary-progressive multiple sclerosis, or progressive-relapsing multiple sclerosis. In some embodiments, the subject is experiencing progression of the multiple sclerosis. In other embodiments, the subject is experiencing a relapse of the multiple sclerosis.

In some embodiments, a composition comprising a compound of Formula I or Formula II (e.g., nicotinamide riboside), and/or a compound of Formula III (e.g., pterostilbene) may be administered to the subject conjointly with a second therapeutic for the treatment of MS. The second therapeutic may be a immunotherapeutic agent (e.g., interferon-beta 1a, interferon-beta 1b, glatiramer acetate, natalizumab, mitoxantrone, fingolimod, teriflunomide, dimethyl fumarate, mycophenolate mofetil, paclitaxel, cyclosporine, corticosteroids, azathioprine, cyclophosphamide, methotrexate, cladribine, 4-aminopyridine, and tizanidine) or a hormone (e.g., estrogen or testosterone).

In certain embodiments of the compositions and methods provided herein, the composition comprises a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)). In some embodiments, the composition comprises a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)). In certain embodiments, the composition comprises both a compound of Formula I or Formula II (e.g., nicotinamide riboside) (e.g., at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside)) and a compound of Formula III (e.g., pterostilbene) (e.g., at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene)).

In certain embodiments, the method comprises administering a plurality of doses of the composition. In some embodiments, at least 7 doses of the composition are administered. In some embodiments, at least 30 doses of the composition are administered. In some embodiments, at least 60 or more doses of the composition are administered. In some embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside). In some embodiments, each dose comprises at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of Formula III (e.g., pterostilbene). In certain embodiments, each dose comprises at least 100 mg, at least 125 mg, at least 150 mg, at least 175 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 525 mg, at least 550 mg, at least 575 mg or at least 600 mg of a compound of Formula I or Formula II (e.g., nicotinamide riboside) at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg or at least 150 mg of a compound of formula III (e.g., pterostilbene).

In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months or for at least 1 year.

In certain embodiments, the composition is formulated for oral delivery. In some embodiments, the composition is formulated as a pill, a tablet, or a capsule. In some embodiments, the composition is administered orally. In certain embodiments, the composition is self-administered.

### DETAILED DESCRIPTION

### General

In some aspects, the methods and compositions disclosed herein relate to treating an autoimmune disease, such as multiple sclerosis. Provided herein are methods and compositions related to treating multiple sclerosis and/or for treating and/or preventing a symptom of multiple sclerosis, by administering to the subject (e.g., orally administering to the subject) a composition comprising a compound of Formula I or Formula II (e.g., nicotinamide riboside), and/or a compound of Formula III (e.g., pterostilbene).

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Administration of a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

The phrase “pharmaceutically-acceptable carrier” as used herein means a pharmaceutically-acceptable material, composition or vehicle, such as a liquid or solid filler, diluent, excipient, or solvent encapsulating material.

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

As used herein, a therapeutic that “prevents” a disorder or condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

### Compositions

Provided herein are pharmaceutical compositions comprising a compound of Formula I or Formula II (e.g., nicotinamide riboside) and/or a compound of Formula III (e.g., pterostilbene).

Nicotinamide riboside is a pyridine-nucleoside form of niacin (i.e., vitamin B3) that serves as a precursor to nicotinamide adenine dinucleotide (NAD⁺). As used herein, “nicotinamide riboside” also includes nicotinamide riboside salts, such as nicotinamide riboside chloride. The chemical structure of nicotinamide riboside is provided below:

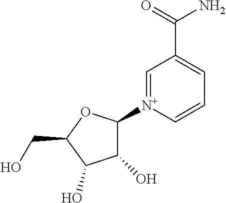 [SMILES: NC(=O)c1ccc[n+]([C@@H]2O[C@H](CO)[C@@H](O)[C@H]2O)c1]

In some embodiments, provided herein are pharmaceutical compositions comprising a compound represented by Formula (I) or a pharmaceutically acceptable salt thereof:

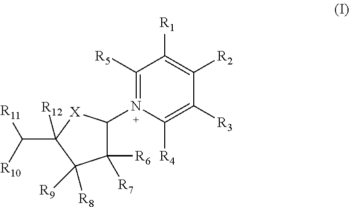

wherein, independently for each occurrence:

R₁, R₂, and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, heteroaralkyl, —OR₁₄, and —N(R₁₄)ₘ;

R₇, R₉, and R₁₀ are selected from —((C₁-C₆)alkylene)N(R₁₄)ₘ, —OR₁₄, and —N(R₁₄)ₘ;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

X is O, S, or N(R₁₄);

m is 2 or 3;

provided that at least one of R₁, R₂, and R₃ is R₁₃.

In some embodiments, R₁ is R₁₃. In some embodiments, R₂ is R₁₃. In some embodiments, R₃ is R₁₃.

In some embodiments, R₁₃ is selected from —OR₁₄, —N(R₁₄)ₘ, —C(O)(R₁₄), —C(O)(OR₁₄), and —C(O)N(R₁₄)ₘ. In some embodiments, R₁₃ is selected from —C(O)(R₁₄), —C(O)(OR₁₄), and —C(O)N(R₁₄)ₘ. In some embodiments, R₁₃ is —C(O)N(R₁₄)ₘ.

In some embodiments, R₇, R₉, and R₁₀ are each independently —OR₁₄ or —N(R₁₄)ₘ. In some embodiments, R₇, R₉, and R₁₀ are —OR₁₄.

In some embodiments, the compound of formula (I) is represented by Formula (II) or a pharmaceutically acceptable salt thereof:

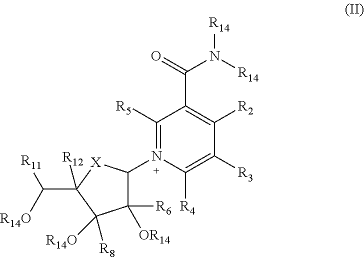

wherein, independently for each occurrence:

R₂ and R₃ are selected from hydrogen, halogen, —CN, —NO₂, —OR₁₄, —N(R₁₄)ₘ, —R₁₃, substituted or unsubstituted (C₁-C₆)alkyl, cycloalkyl, heterocycloalkyl, aryl, heteroaryl, aralkyl, and heteroaralkyl;

R₄ and R₅ are selected from hydrogen, halogen, —CN, —NO₂, —OR₁₄, —N(R₁₄)ₘ, substituted or unsubstituted (C₁-C₆)alkyl, cycloalkyl, heterocycloalkyl, aryl, heteroaryl, aralkyl, and heteroaralkyl;

R₆, R₈, R₁₁, and R₁₂ are selected from hydrogen, —OR₁₄, —N(R₁₄)ₘ, substituted or unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl;

R₁₃ is selected from —OR₁₄, —N(R₁₄)ₘ, —C(O)(R₁₄), —C(O)(OR₁₄), —C(O)N(R₁₄)ₘ, —S(O)₂(OR₁₄), —S(O)OR₁₄, and —S(O)₂N(R₁₄)ₘ;

R₁₄ is selected from hydrogen, (C₁-C₆)alkyl, cycloalkyl, heterocycloalkyl, aryl, heteroaryl, aralkyl, and heteroaralkyl; and

m is 2 or 3.

In some embodiments of the compounds of formula (I) or (II), R₁, R₂, and R₃ are each independently, if present, selected from hydrogen, halogen, —CN, —NO₂, —OR₁₄, —N(R₁₄)ₘ, —R₁₃, and substituted or unsubstituted (C₁-C₆)alkyl. In some embodiments, R₁, R₂, and R₃ are each independently, if present, selected from hydrogen, —OR₁₄, —N(R₁₄)ₘ, and unsubstituted (C₁-C₆)alkyl. In some embodiments, R₁, R₂, and R₃ are each independently, if present, selected from substituted or unsubstituted (C₁-C₆)alkyl, cycloalkyl, heterocycloalkyl, aryl, heteroaryl, aralkyl, and heteroaralkyl. In some embodiments, R₁, R₂, and R₃ are each independently, if present, hydrogen.

In some embodiments of the compounds of formula (I) or (II), R₄ and R₅ are each independently selected from hydrogen, halogen, —CN, —NO₂, —OR₁₄, —N(R₁₄)ₘ, and substituted or unsubstituted (C₁-C₆)alkyl. In some embodiments, R₄ and R₅ are each independently selected from hydrogen, —OR₁₄, —N(R₁₄)ₘ, and unsubstituted (C₁-C₆)alkyl. In some embodiments, R₄ and R₅ are each independently selected from substituted or unsubstituted (C₁-C₆)alkyl, cycloalkyl, heterocycloalkyl, aryl, heteroaryl, aralkyl, and heteroaralkyl. In some embodiments, R₄ and R₅ are each hydrogen.

In some embodiments of the compounds of formula (I) or (II), R₆, R₈, R₁₁, and R₁₂ are selected from hydrogen, —OR₁₄, —N(R₁₄)ₘ, unsubstituted (C₁-C₆)alkyl, —((C₁-C₆)alkylene)N(R₁₄)ₘ, —C(O)((C₁-C₆)alkylene)N(R₁₄)ₘ, cycloalkyl, heterocycloalkyl, aryl, heteroaryl, aralkyl, and heteroaralkyl. In some embodiments, R₆, R₈, R₁₁, and R₁₂ are each independently selected from hydrogen, —OR₁₄, —N(R₁₄)ₘ, unsubstituted (C₁-C₆)alkyl, —((C₁-C₆)alkylene)N(R₁₄)ₘ, and —C(O)((C₁-C₆)alkylene)N(R₁₄)ₘ. In some embodiments, R₆, R₈, R₁₁, and R₁₂ are each independently selected from hydrogen, —OR₁₄, and —N(R₁₄)ₘ. In some embodiments, R₆, R₈, R₁₁, and R₁₂ are each independently selected from unsubstituted (C₁-C₆)alkyl, cycloalkyl, heterocycloalkyl, aryl, heteroaryl, aralkyl, and heteroaralkyl. In some embodiments, R₆, R₈, R₁₁, and R₁₂ are each hydrogen.

In some embodiments, R₇, R₉, and R₁₀ are each independently —OR₁₄ or —N(R₁₄)ₘ. In some embodiments, R₇, R₉, and R₁₀ are each —OR₁₄. In some embodiments, R₇, R₉, and R₁₀ are each —OH.

In some embodiments of the compounds of formula (I) or (II), R₁₄ is hydrogen or (C₁-C₆)alkyl.

In some embodiments of the compounds of formula (I) or (II), X is O or N(R₁₄). In some embodiments, X is O.

In some embodiments of the compounds of formula (I) or (II), the compound is

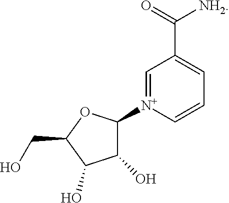 [SMILES: NC(=O)c1ccc[n+]([C@@H]2O[C@H](CO)[C@@H](O)[C@H]2O)c1]

Pterostilbene is a stilbenoid and an analog of polyphenol reservatrol that has better bioavailability due to the presence of two methoxy groups that allow it to have increased lipophilic and oral absorption as well as a longer half-life due to reduced oxidation. The chemical structure of pterostilbene is provided below:

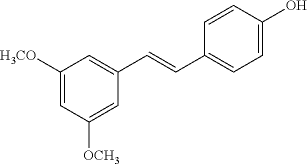 [SMILES: COc1cc(/C=C/c2ccc(O)cc2)cc(OC)c1]

In some embodiments, provided herein are pharmaceutical compositions comprising a compound represented by Formula (III) or a pharmaceutically acceptable salt thereof:

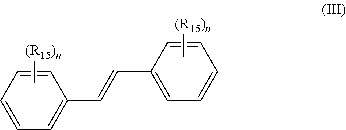

wherein, independently for each occurrence:

R₁₅ is selected from halogen, —CN, —NO₂, —OR₁₆, —N(R₁₆)ₚ, —S(O)₂(OR₁₆), —S(O)OR₁₆, substituted or unsubstituted (C₁-C₆)alkyl, cycloalkyl, heterocycloalkyl, aryl, heteroaryl, aralkyl, and heteroaralkyl;

R₁₆ is selected from hydrogen, (C₁-C₆)alkyl, cycloalkyl, heterocycloalkyl, aryl, heteroaryl, aralkyl, and heteroaralkyl;

n is an integer from 0 to 5; and

p is 2 or 3;

provided that at least one n is 1; and at least one R₁₅ is —OR₁₆;

provided that the compound of formula (III) is not

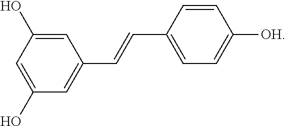 [SMILES: Oc1ccc(/C=C/c2cc(O)cc(O)c2)cc1]

In some embodiments of the compounds of formula (III), R₁₅ is selected from, halogen, —CN, —NO₂, —OR₁₆, —N(R₁₆)ₚ, and substituted or unsubstituted (C₁-C₆)alkyl. In some embodiments, R₁₅ is selected from —OR₁₆, —N(R₁₆)ₚ, and unsubstituted (C₁-C₆)alkyl. In some embodiments, R₁₅ is selected from substituted or unsubstituted (C₁-C₆)alkyl, cycloalkyl, heterocycloalkyl, aryl, heteroaryl, aralkyl, and heteroaralkyl. In some embodiments, R₁₅ is —OR₁₆. In some embodiments, R₁₅ is —OR₁₆; and R₁₆ is hydrogen or (C₁-C₆)alkyl. In some embodiments, R₁₅ is —OR₁₆, and R₁₆ is (C₁-C₆)alkyl, cycloalkyl, heterocycloalkyl, aryl, heteroaryl, aralkyl, and heteroaralkyl. In some embodiments, R₁₅ is —OR₁₆, and R₁₆ is (C₁-C₆)alkyl. In some embodiments, R₁₅ is —OR₁₆, and R₁₆ is (C₁-C₆)alkyl, cycloalkyl, or heterocycloalkyl.

In some embodiments, n is 1, 2, or 3. In some embodiments, n is 1 or 2.

In some embodiments, p is 2. In some embodiments, p is 3.

In one aspect, the provided herein are pharmaceutically acceptable compositions which comprise a therapeutically-effective amount of one or more of the compounds described herein (e.g., nicotinamide riboside and/or pterostilbene), formulated together with one or more pharmaceutically acceptable carriers (additives) and/or diluents. In another aspect, the agents described herein can be administered as such, or administered in mixtures with pharmaceutically acceptable carriers and can also be administered in conjunction with other agents. Conjunctive therapy thus includes sequential, simultaneous and separate, or co-administration of one or more compounds of the invention, wherein the therapeutic effects of the first administered has not entirely disappeared when the subsequent compound is administered.

As described in detail below, the pharmaceutical compositions described herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; or (3) sublingually.

In some embodiments, the composition comprises additional agents. For example, the composition may comprise a nutritional agent, such as an antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The formulations of the compounds described herein may be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the agent which produces a therapeutic effect.

In certain embodiments, a formulation described herein comprises an excipient, including, but not limited to, cyclodextrins, liposomes, micelle forming agents, e.g., bile acids, and polymeric carriers, e.g., polyesters and polyanhydrides; and an agent of the invention. In some embodiments, an aforementioned formulation renders orally bioavailable an agent of the invention. Methods of preparing these formulations or compositions may include the step of bringing into association a compound of the invention with the carrier and, optionally, one or more accessory ingredients.

Liquid dosage forms for oral administration of the formulations provided herein include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations provided herein suitable for oral administration may be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound of the invention may also be administered as a bolus, electuary, or paste.

In solid dosage forms of the invention for oral administration (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions described herein may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Pharmaceutical compositions provided herein suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

### Therapeutic Methods

Provided herein are methods and compositions related to treating an autoimmune disease (e.g., multiple sclerosis) in a subject by administering to the subject (e.g., orally administering to the subject) a composition comprising a compound of Formula I or Formula II (e.g., nicotinamide riboside), and/or a compound of Formula III (e.g., pterostilbene).

Multiple sclerosis (MS), also known as disseminated sclerosis or encephalomyelitis disseminata) is an inflammatory autoimmune disease of the central nervous system (CNS). The cause of MS appears to be a combination of immunological, genetic and environmental factors. MS is a chronic demyelinating disease, which primarily affects young adults and is characterized by a highly variable course. The heterogeneous presentation of MS is characterized by a variety of clinical problems arising from multiple regions of demyelination and inflammation along axonal pathways. The signs and symptoms of MS are determined by the location of the affected regions. Mostly, the disease begins in the third or fourth decade of life. Its initial course is characterized by acute episodes of neurological dysfunction, such as decreased vision, followed by subsequent recovery. This course is known as relapsing-remitting MS. Over time, the improvement after attacks may be incomplete and the relapsing-remitting course may evolve into one of increasing progression of disability, termed secondary progressive MS.

In certain embodiments, the multiple sclerosis is relapsing-remitting multiple sclerosis. In certain embodiments, the multiple sclerosis is secondary-progressive multiple sclerosis. In certain embodiments, the multiple sclerosis is primary-progressive multiple sclerosis. In certain embodiments, the multiple sclerosis is progressive-relapsing multiple sclerosis. In certain embodiments, the subject has a mild form of any one of the foregoing subtypes of MS. In certain embodiments, the subject has a moderate form of any one of the foregoing subtypes of MS. In certain embodiments, the subject has an aggressive form of any one of the foregoing subtypes of MS.

Relapsing-remitting MS is characterized by unpredictable relapses followed by periods of months to years of remission, with no new signs of disease activity. Deficits that occur during attacks may either resolve or leave sequelae, the latter in about 40% of attacks and being more common the longer a person has had the disease. This describes the initial course of 80% of individuals with MS. The relapsing-remitting subtype usually begins with a clinically isolated syndrome (CIS). In CIS, a person has an attack suggestive of demyelination but does not fulfill the criteria for multiple sclerosis; 30% to 70% of persons experiencing CIS go on to develop MS. Provided herein are methods and compositions for treating a subject that has experiences CIS, but has not been diagnosed with MS by administering to the subject (e.g., orally administering to the subject) a composition comprising a compound of Formula I or Formula II (e.g., nicotinamide riboside), and/or a compound of Formula III (e.g., pterostilbene).

Secondary-progressive MS occurs in around 65% of those with initial relapsing-remitting MS, who eventually have progressive neurologic decline between acute attacks without any definite periods of remission. Occasional relapses and minor remissions may appear. The median length of time between disease onset and conversion from relapsing-remitting to secondary progressive MS is 19 years.

Primary-progressive MS occurs in approximately 10-20% of individuals, with no remission after the initial symptoms. It is characterized by progression of disability from onset, with no, or only occasional and minor, remissions and improvements. The usual age of onset for the primary progressive subtype is later than of the relapsing-remitting subtype, but similar to the age that secondary-progressive MS usually begins in relapsing-remitting MS, around 40 years of age.

Progressive-relapsing MS describes those individuals who, from onset, have a steady neurologic decline but also have clear superimposed attacks. This is the least common form of MS.

The subject may be male or female. In some embodiments, the subject is an adult (i.e., 18 years of age or older). The subject may be pediatric (i.e., less than 18 years of age). In some embodiments, the subject is a mammal, preferably, a human.

Provided herein are methods and compositions related to treating multiple sclerosis (e.g., a subtype of multiple sclerosis disclosed herein) and/or for treating, preventing, and/or improving a symptom of multiple sclerosis by administering (e.g., orally administering to the subject) a composition comprising a compound of Formula I or Formula II (e.g., nicotinamide riboside), and/or a compound of Formula III (e.g., pterostilbene). Symptoms of multiple sclerosis may include, but are not limited to, fatigue, walking (gait) difficulties, numbness or tingling, spasticity, weakness, vision problems, dizziness or vertigo, bladder dysfunction, constipation, loss of control of bowels, chronic pain, depression, mood changes, dysarthria, dysphonia, dysphagia, seizures, tremor, pruritis, headache, hearing loss, and problems with respiration.

The various methods disclosed herein can be methods for improving walking, vision, balance, cognition, or other symptoms in a subject, such as a subject with multiple sclerosis, and/or methods for improving multiple sclerosis functional composite (MSFC), EDSS, or MSSS scores in a subject, such as a subject with multiple sclerosis. Thus, in certain embodiments, the methods of treatment disclosed herein include methods for stabilizing or improving disability in a patient, whereby the patient's disability score (as measured by either of these tests or another suitable test) after a therapy (e.g., after about one month, about three months, after about six months, after about nine months, after about one year, after about two years, or after about three years) comprising administration of a composition disclosed herein is at least about 10%, at least about 25%, at least about 40%, at least about 50%, or even at least about 60% higher relative to a control patient not receiving a therapy disclosed herein. Alternatively, the patient's disability score (as measured by either of these tests or another suitable test) after a therapy (e.g., after about one month, about three months, after about six months, after about nine months, after about one year, after about two years, or after about three years) comprising administration of a composition disclosed herein is at least about 2%, at least about 5%, at least about 10%, at least about 25%, at least about 40%, at least about 50%, or even at least about 60% higher than the earlier assessment.

Improvements in cognition outcomes associated with MS therapy, whether slowing of cognitive decline, stabilization of cognitive decline, or improvement of cognitive function, can be assessed using the PASAT (e.g., PASAT 2 or PASAT 3) or SDMT test, or alternatively the MS-COG test (see Erlanger et al., J Neuro Sci 340: 123-129 (2014)). Thus, in certain embodiments, the methods of treatment disclosed herein include methods for stabilizing or improving cognition in a patient, whereby the patient's cognition outcome after therapy (i.e., a therapy comprising administration of a composition disclosed herein) is at least about 2%, at least about 5%, at least about 10%, at least about 25%, at least about 50%, or even at least about 75% higher relative to a control patient not receiving the therapy, e.g., as measured by any of the preceding tests. Alternatively, the patient's cognition outcome after about one month, about three months, after about six months, after about nine months, after about one year, after about two years, or after about three years of therapy (i.e., a therapy comprising administration of a composition disclosed herein) may be at least about 2%, at least about 5%, at least about 10%, at least about 25%, at least about 50%, or even at least about 75% higher than the earlier assessment, e.g., as measured by any of the preceding tests at different times.

For example, a subject who scores below 50 on PASAT (and optionally if such low score is verified upon a second subsequent test, such as within one week to one month of the first) may be deemed to have cognitive disability. For such a patient not already receiving therapy (i.e., a therapy comprising administration of a composition disclosed herein), the subject demonstrating the cognitive disability may commence treatment with a composition disclosed herein. In certain embodiments, the cognitive test may be repeated (e.g., at about six months from the start of treatment) to assess whether the treatment slowed or halted any further worsening in cognitive performance, e.g., as measured by the PASAT test. In certain such embodiments, the patient's score may increase by at least 3 points over the course of six to twelve months of therapy.

Clinically, MS can be assessed and monitored using any of a number of structural (anatomical) and functional tests, including, without limitation: magnetic resonance imaging (MRI); Paced Serial Addition Test (PASAT); symbol digit modalities test (SDMT); expanded disability status score (EDSS); multiple sclerosis functional composite (MSFC); 25-foot walk test; 9-hole peg test; low contrast visual acuity; MS Quality of Life; Modified Fatigue Impact Scale; Beck Depression Inventory; 7/24 Spatial Recall Test; Benton Forms F & G; Buschke Selective Reminding Test; Verbal Paired Associates; Word List Generation. Recently, the PASAT test of cognitive function has come under criticism by some for its test-retest reliability and practice effect whereby one naturally improves over time with repeated test taking. Polman C H et al., Neurology 74 Suppl 3: S8-15 (2010). In some embodiments, assessment of MacDonald dissemination in space and time finds use in the present methods. For example, for dissemination in space, lesion imaging, such as, by way of illustration, Barkhof-Tintore MR imaging criteria, may be used. For instance, the following criteria can be evaluated: (1) at least one gadolinium-enhancing lesion or 9 T2 hyperintense lesions; (2) at least one infratentorial lesion; (3) at least one juxtacortical lesion; (4) at least 3 periventricular lesions; and (5) a spinal cord lesion. Such imaging criteria can optionally be used in combination with evaluation for immunoglobulin abnormalities in the cerebrospinal fluid (CSF), for example. For dissemination in time, MR imaging can also be used. For example, if an MR imaging scan of the brain performed at ≥3 months after an initial clinical event demonstrates a new gadolinium-enhancing lesion, this may indicate a new CNS inflammatory event, because the duration of gadolinium enhancement in MS is usually less than 6 weeks. If there are no gadolinium-enhancing lesions but a new T2 lesion (presuming an MR imaging at the time of the initial event), a repeat MR imaging scan after another 3 months may be needed with demonstration of a new T2 lesion or gadolinium-enhancing lesion. In various embodiments, any one or more of these structural (anatomical) and functional tests may be used in conjunction with the present invention (e.g., to assess the effectiveness of a disclosed treatment method).

Provided herein are methods and compositions related to treating multiple sclerosis in a subject by administering to the subject (e.g., orally administering to the subject) a composition comprising a compound of Formula I or Formula II (e.g., nicotinamide riboside), and/or a compound of Formula III (e.g., pterostilbene) conjointly with a second therapeutic for MS. In some embodiments, the second therapeutic is an immunotherapeutic agent. At least in connection with MS, such agents are sometimes referred to as disease-modifying therapies or disease-modifying therapeutics (DMTs).

The term “immunotherapeutic agent” as used herein refers to a compound with an objectively measurable effect on at least one aspect of the immune system or an immune response. In certain embodiments, the immunotherapeutic agent is immunosuppressive, i.e., it exerts an objectively measurable inhibitory effect on at least one aspect of the immune system or an immune response. In certain embodiments, the immunotherapeutic agent is anti-inflammatory. In certain embodiments, the immunotherapeutic agent is a small molecule (molecular weight less than or equal to about 1.5 kDa) pharmaceutical compound or composition. In certain embodiments, the immunotherapeutic agent is a biological compound or composition, e.g., an antibody, peptide, nucleic acid, etc.

In certain embodiments, the immunotherapeutic agent is selected from dimethyl fumarate (Tecfidera®; BG-12), fingolimod (Gilenya®), glatiramer acetate (Copaxone®, for example “longer-lasting” 40 mg/ml or 20 mg/ml versions), interferon beta-1a (Avonex® or Rebif®), interferon beta-1b (Betaseron® or Extavia®), mitoxantrone (Novantrone®), natalizumab (Tysabri®), and teriflunomide (Aubagio®), mycophenolate mofetil, paclitaxel, cyclosporine, corticosteroids (e.g., prednisone, methylprenisolone), azathioprine, cyclophosphamide, methotrexate, cladribine, 4-aminopyridine, and tizanidine.

In certain embodiments, the immunotherapeutic agent is dimethyl fumarate (Tecfidera®; BG-12). In certain embodiments, the immunotherapeutic agent is fingolimod (Gilenya®). In certain embodiments, the immunotherapeutic agent is glatiramer acetate (Copaxone®). In certain embodiments, the immunotherapeutic agent is interferon beta-1a (Avonex® or Rebif®). In certain embodiments, the immunotherapeutic agent is interferon beta-1b (Betaseron® or Extavia®). In certain embodiments, the immunotherapeutic agent is mitoxantrone (Novantrone®). In certain embodiments, the immunotherapeutic agent is natalizumab (Tysabri®). In certain embodiments, the immunotherapeutic agent is teriflunomide (Aubagio®).

In some embodiments, the second therapeutic is a hormone, such as estrogen or testosterone. The estrogen may be estradiol, estrone and estriol. In some embodiments, the subject is undergoing hormone therapy. For example, the subject may be female, and receiving estrogen-based hormone therapy. Conversely, the subject may be male, and receiving a testosterone-based hormone therapy. For more details about hormone therapy in the treatment of MS, please see Sicotte et al. Testosterone treatment in multiple sclerosis: a pilot study. Arch Neurol. 2007 May; 64(5):683-8 and Sicotte et al. Treatment of multiple sclerosis with the pregnancy hormone estriol. Ann Neurol. 2002; 52:421-428, each of which is hereby incorporated by reference in their entireties.

In certain embodiments, the subject is already receiving a disease-modifying therapeutic or hormone therapy. In this circumstance, the subject can continue to receive the disease-modifying therapeutic or hormone while beginning treatment with a composition comprising a compound of Formula I or Formula II (e.g., nicotinamide riboside), and/or a compound of Formula III (e.g., pterostilbene).

In certain embodiments, the subject is receiving an immunotherapeutic agent or hormone and experiencing a relapse or progression of the multiple sclerosis. For example, a subject may experience a relapse or progression while on a maintenance dose of a DMT. Such subject can then begin concurrent treatment with a composition (e.g., a composition comprising a compound of Formula I or Formula II (e.g., nicotinamide riboside), and/or a compound of Formula III (e.g., pterostilbene) in accordance with any of the various methods disclosed herein, e.g., to reduce the frequency and/or severity of relapses or to slow progression of the disease (e.g., as determined by assessment of one or more of walking, vision, balance, cognition, or other symptoms of the condition, e.g., as measured according to the Expanded Disability Severity Scale (EDSS) and/or the multiple sclerosis functional composite (MSFC)). Thus, the various embodiments of the methods disclosed herein can be methods for improving walking, vision, balance, cognition, or other symptoms in a subject, such as a subject with multiple sclerosis, and/or methods for improving EDSS or MSFC scores in a subject, such as a subject with multiple sclerosis.

In certain embodiments, the subject is receiving an immunotherapeutic agent or hormone and experiencing a relapse of the multiple sclerosis. For example, a subject may experience a relapse while on a maintenance dose of a DMT. Such subject can then begin concurrent treatment with a composition disclosed herein (e.g., a composition comprising a compound of Formula I or Formula II (e.g., nicotinamide riboside), and/or a compound of Formula III (e.g., pterostilbene) in accordance with a method of the present invention, e.g., to reduce the frequency and/or severity of relapses.

Actual dosage levels and administration regimen of the compositions disclosed herein may be varied so as to obtain an amount of a compound of Formula I or Formula II (e.g., nicotinamide riboside) and/or a compound of Formula III (e.g., pterostilbene) that is effective to achieve the desired therapeutic response for a particular patient, composition, and mode of administration, without being toxic to the patient. In some embodiments, the subject continuously self-administers the compounds disclosed herein. In other embodiments, the subject may take a compound disclosed herein as needed.

In some embodiments, administration of the composition comprises administration of the composition in one or more dose(s). In some embodiments, administration of the composition comprises administration of the composition in one or more, five or more, ten or more, twenty or more, thirty or more, forty or more, fifty or more, one hundred or more, or one thousand or more dose(s). In some embodiments, the dose comprises at least 25 mg, at least 50 mg, at least 75 mg, at least 100 mg, at least 125 mg, at least 150 mg, at least 200 mg, at least 225 mg, at least 250 mg, at least 275 mg, at least 300 mg, at least 325 mg, at least 350 mg, at least 375 mg, at least 400 mg, at least 425 mg, at least 450 mg, at least 475 mg, at least 500 mg, at least 550 mg, at least 600 mg, at least 650 mg, at least 700 mg, at least 750 mg, at least 800 mg, or at least 850 mg of a compound of Formula I or Formula II (e.g., nicotinamide riboside). In some embodiments, the dose comprises at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 90 mg, at least 95 mg, at least 100 mg, at least 110 mg, at least 120 mg, at least 130 mg, at least 140 mg, at least 150 mg, at least 160 mg, least 170 mg, at least 180 mg, at least 190 mg, at least 200 mg, or at least 250 mg of a compound of formula III (e.g., pterostilbene).

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating multiple sclerosis in a subject comprising administering to the subject a composition comprising nicotinamide riboside.

2. The method of claim 1, wherein the composition further comprises pterostilbene.

3. The method of claim 1 or 2, wherein the multiple sclerosis is relapsing-remitting multiple sclerosis.

4. The method of claim 1 or 2, wherein the multiple sclerosis is secondary-progressive multiple sclerosis.

5. The method of claim 1 or 2, wherein the multiple sclerosis is primary-progressive multiple sclerosis.

6. The method of claim 1 or 2, wherein the multiple sclerosis is progressive-relapsing multiple sclerosis.

7. The method of any one of the preceding claims, wherein the subject is experiencing progression of the multiple sclerosis.

8. The method of any one of the preceding claims, wherein the subject is experiencing a relapse of the multiple sclerosis.

9. A method of treating and/or preventing a symptom of multiple sclerosis in a subject comprising administering to the subject a composition comprising nicotinamide riboside.

10. The method of claim 9, wherein the composition further comprises pterostilbene.

11. The method of claim 9 or 10, wherein the symptom is fatigue, walking (gait) difficulties, numbness or tingling, spasticity, weakness, vision problems, dizziness or vertigo, bladder dysfunction, constipation, loss of control of bowels, chronic pain, depression, mood changes, dysarthria, dysphonia, dysphagia, seizures, tremor, pruritus, headache, hearing loss, and problems with respiration.

12. The method of any one of claims 9 to 11, wherein the subject has relapsing-remitting multiple sclerosis.

13. The method of any one of claims 9 to 11, wherein the subject has secondary-progressive multiple sclerosis.

14. The method of any one of claims 9 to 11, wherein the subject has primary-progressive multiple sclerosis.

15. The method of any one of claims 9 to 11, wherein the subject has progressive-relapsing multiple sclerosis.

16. The method of any one of the preceding claims, wherein the method comprises conjointly administering a second therapeutic to the subject.

17. The method of claim 16, wherein the second therapeutic is an immunotherapeutic agent.

18. The method of claim 17, wherein the immunotherapeutic agent is selected from interferon-beta 1a, interferon-beta 1b, glatiramer acetate, natalizumab, mitoxantrone, fingolimod, teriflunomide, dimethyl fumarate, mycophenolate mofetil, paclitaxel, cyclosporine, corticosteroids, azathioprine, cyclophosphamide, methotrexate, cladribine, 4-aminopyridine, and tizanidine

19. The method of claim 16, wherein the second therapeutic is a hormone.

20. The method of claim 19, wherein the hormone is estrogen.

21. The method of claim 19, wherein the hormone is testosterone.

22. The method of any one of claims 1 to 21, wherein the administration of the composition comprises administering one or more doses of the composition.

23. The method of claim 22, wherein each dose of the composition comprises at least 200 mg of nicotinamide riboside.

24. The method of claim 22, wherein each dose of the composition comprises at least 250 mg of nicotinamide riboside.

25. The method of claim 22, wherein each dose of the composition comprises at least 300 mg of nicotinamide riboside.

26. The method of claim 22, wherein each dose of the composition comprises at least 350 mg of nicotinamide riboside.

27. The method of claim 22, wherein each dose of the composition comprises at least 400 mg of nicotinamide riboside.

28. The method of claim 22, wherein each dose of the composition comprises at least 450 mg of nicotinamide riboside.

29. The method of claim 22, wherein each dose of the composition comprises at least 500 mg of nicotinamide riboside.

30. The method of claim 22, wherein each dose of the composition comprises at least 550 mg of nicotinamide riboside.

31. The method of any one of claims 22 to 30, wherein each dose of the composition comprises at least 15 mg of pterostilbene.

32. The method of any one of claims 22 to 30, wherein each dose of the composition comprises at least 25 mg of pterostilbene.

33. The method of any one of claims 22 to 30, wherein each dose of the composition comprises at least 50 mg of pterostilbene.

34. The method of any one of claims 22 to 30, wherein each dose of the composition comprises at least 75 mg of pterostilbene.

35. The method of any one of claims 22 to 30, wherein each dose of the composition comprises at least 100 mg of pterostilbene.

36. The method of any one of claims 22 to 30, wherein each dose of the composition comprises at least 125 mg of pterostilbene.

37. The method of any one of claims 22 to 30, wherein each dose of the composition comprises at least 150 mg of pterostilbene.

38. The method of any one of claims 22 to 37, wherein two or more doses of the composition are administered.

39. The method of any one of claims 22 to 38, wherein thirty or more doses of the composition are administered.

40. The method of any one of claims 22 to 39, wherein fifty or more doses of the composition are administered.

41. The method of any one of claims 22 to 40, wherein one hundred or more doses of the composition are administered.

42. The method of any one of claims 22 to 41, wherein the dose of the composition is administered at least once a week.

43. The method of any one of claims 22 to 41, wherein the dose is administered at least twice a week.

44. The method of any one of claims 22 to 41, wherein the dose is administered at least three times a week.

45. The method of any one of claims 22 to 41, wherein the dose is administered at least once a day.

46. The method of any one of claims 22 to 41, wherein the dose is administered at least twice a day.

47. The method of any one of claims 42 to 46, wherein the doses are administered for at least 7 days.

48. The method of any one of claims 42 to 46, wherein the doses are administered for at least 30 days.

49. The method of any one of claims 42 to 46, wherein the doses are administered for at least 60 days.

50. The method of any one of claims 42 to 46, wherein the doses are administered for at least 90 days.

51. The method of any one of claims 1 to 50, wherein the composition is formulated as a pill, a tablet, or a capsule.

52. The method of any one of claims 1 to 51, wherein the composition is administered orally.

53. The method of any one of claims 1 to 52, wherein the composition is self-administered.
